(19) United States
(12) Patent Application Publication
(10) Pub. No.: US 2023/0365988 A1
(43) Pub. Date: Nov. 16, 2023

(54) MOUSE MODEL FOR BIO-IMAGING OF INFLAMMATORY SIGNALS, PREPARATION METHOD THEREFOR AND USE THEREOF

(71) Applicants: KOREA MOUSE PHENOTYPING CENTER, Soul (KR); NATIONAL CANCER CENTER, Goyang-si (KR); SEOUL NATIONAL UNIVERSITY R&DB FOUNDATION, Seoul (KR)

(72) Inventors: Je Kyung Seong, Seoul (KR); Ho Lee, Goyang-si (KR); Seung Hyun Oh, Incheon (KR); Min Woo Kim, Seoul (KR); Yoon Jeon, Goyang-si (KR)

(73) Assignees: KOREA MOUSE PHENOTYPING CENTER, Soul (KR); NATIONAL CANCER CENTER, Goyang-si (KR); SEOUL NATIONAL UNIVERSITY R&DB FOUNDATION, Seoul (KR)

(21) Appl. No.: 18/033,442

(22) PCT Filed: Nov. 11, 2021
(86) PCT No.: PCT/KR2021/016375

§ 371 Date: Apr. 24, 2023

(30) Foreign Application Priority Data
Nov. 27, 2020 (KR) 10-2020-0161859

(51) Int. Cl. C12N 15/85

(52) U.S. Cl. CPC C12N 15/8509; A01K 2267/0368; A01K 2227/105; C12N 2015/8527

(57) ABSTRACT

The present invention relates to a mouse model for bio-imaging of inflammation, a preparation method therefor and a use thereof. Particularly, by using the mouse model, which is made by crossing a mouse model with a mouse expressing Cre recombinase specifically in certain tissues and cells, certain tissue- and cell-specific inflammatory signals desired by a researcher can be evaluated in vitro and the progress of inflammatory changes over time without the autopsy of living animals can be identified, and thus the present invention can be used throughout the field of life science research including experimental animal science, molecular biology and the like, and can also be used as a model for efficacy evaluation of anti-inflammatory candidates in the industrial area such as new drug or health functional food development and the like.

### BACKGROUND OF THE INVENTION

### 1. Field of the Invention

The present invention relates to a mouse model for bio-imaging of inflammation, a preparation method therefor and a use thereof.

### 2. Description of the Related Art

Inflammation is a defense mechanism induced by infection or body damage, etc., and is a complex biological response that is involved by various cells and regulated by multiple cytokines and signal transduction pathways. It is known that the product of the inflammatory response induces damage to surrounding tissues, and that the damage of these tissues is associated with the development of various diseases.

Recently, the incidence of diseases accompanied by chronic inflammation has increased not only in Korea but also around the world, and in most cases, the above diseases are treated by methods of suppressing inflammation. However, the mechanism by which inflammation is induced varies depending on the characteristics and etiology of organs and tissues in which each disease develops, and the degree to which the drug reaches varies, so the strategy of controlling inflammation should be considered differently for each disease.

Drugs to treat inflammation are largely divided into steroidal anti-inflammatory drugs (SAIDs) and non-steroidal anti-inflammatory drugs (NSAIDs/NAIDs).

Steroidal anti-inflammatory drugs act on glucocorticoid receptors to suppress the inflammation-related gene expression, thereby exhibiting anti-inflammatory effects. Steroidal anti-inflammatory drugs have stronger effects than non-steroidal anti-inflammatory drugs, but have strong side effects, so long-term administration is difficult.

Non-steroidal anti-inflammatory drugs inhibit COX (cyclooxygenase), which synthesizes prostaglandin, a mediator of inflammation, and exhibit antipyretic, analgesic and anti-inflammatory effects, and are one of the most commonly prescribed drugs in the world, but have side effects such as blood coagulation disorders, bleeding, gastrointestinal disorders, or ulcers.

Accordingly, anti-cytokine drugs that inhibit the action of inflammatory cytokines such as TNF-α (tumor necrosis factor-α), IL-1 (interleukin-1) or IL-6 (interleukin-6) have recently been developed and used. However, since these are antibody-based protein drugs, they have disadvantages such as being impossible to administer orally, having side effects of inhibiting the action of the cytokine throughout the body in addition to the inflammatory site, and being too expensive. Therefore, it is necessary to develop an anti-inflammatory agent that overcomes the side effects and disadvantages described above, and accordingly, the demand for animal models that can more effectively screen anti-inflammatory agents and evaluate anti-inflammatory efficacy is also increasing.

Existing inflammatory disease animal models basically evaluate the degree of inflammatory cell infiltration or damage of the tissue by conducting histopathological evaluation in the tissue to evaluate the degree of inflammation, or the level of inflammation-related signaling proteins or inflammatory cytokines.

However, in the case of histopathological evaluation of inflammation, only the degree of inflammation at the time of autopsy is reflected in the evaluation because the tissue must be collected by autopsy of the animal, and there is a limit that the change in the inflammatory response before the autopsy is not known. In addition to the above evaluation, depending on the inflammatory disease model, if there are usable hematological inflammatory markers or when weight changes or behavioral changes are accompanied, these indicators may be additionally used for the inflammation evaluation. Since these indicators can be measured several times from the time of inflammation induction to the time of autopsy, it has the advantage of being able to indirectly confirm the changes in tissue inflammation over time. However, hematologic indicators, weight changes, and behavioral changes are lagging indicators that are affected and changed after tissue inflammation occurs, so the degree of inflammation at each point in time is not always reflected.

In view of this, the present inventors attempted to develop a mouse model for bio-imaging of inflammation capable of evaluating the degree of inflammation in a mouse in vitro at a desired time point without autopsy.

Accordingly, the present inventors prepared a mouse model expressing luciferase in accordance with the activation of NF-κ inflammatory signals, and mated the model with a mouse expressing Cre recombinase specifically in certain cells and tissues to establish a mouse model capable of cell/tissue-specific bio-imaging of inflammatory signals. And the present inventors completed the present invention by confirming that the mouse model is effective as a mouse model for bio-imaging of inflammatory signals by confirming that the signal by luciferase increases in vitro when inflammation is induced in the mouse model.

### SUMMARY OF THE INVENTION

It is an object of the present invention to provide a mouse model for bio-imaging of inflammatory signals capable of bio-imaging by measuring cell/tissue-specific inflammatory signals several times in vitro without autopsy of a mouse to confirm the degree of inflammation, a preparation method therefor and an anti-inflammatory substance screening method using the same.

To achieve the above object, the present invention provides a method for preparing a mouse model for bio-imaging of inflammatory signals comprising the following steps:
1) a step of preparing a targeting vector by inserting a gene cassette including a nucleotide sequence encoding NF-κB RE (nuclear factor kappa-light-chain-enhancer of activated B cells response element) and a reporter gene into a mouse ROSA26 locus;
2) a step of preparing a mouse embryonic stem cell clone by inserting the targeting vector prepared in step 1) into mouse embryonic stem cells;
3) a step of inserting the mouse embryonic stem cell clone prepared in step 2) into the blastocyst isolated from wild-type mice;
4) a step of implanting the blastocyst into which the clone of step 3) is inserted into the uterus of a surrogate mouse; and
5) a step of preparing a heterozygous mouse by mating the mouse born from the surrogate mouse of step 4) with a wild-type mouse.

The present invention also provides a mouse model for bio-imaging of inflammatory signals prepared by method for preparing a mouse model for bio-imaging of inflammatory signals.

The present invention also provides a method for screening an anti-inflammatory substance comprising the following steps:
1) a step of treating a test substance to a mouse prepared by mating the mouse model of claim 7 with a genetically engineered mouse expressing Cre recombinase specifically in certain cells and tissues;
2) a step of measuring the activity level of NF-κ (nuclear factor kappa-light-chain-enhancer of activated B cells) in the mouse treated with the test substance; and
3) a step of selecting a test substance that reduced the activity level of NF-κ compared to the control group.

### Advantageous Effect

By using the mouse model, which is made by mating a mouse model with a mouse expressing Cre recombinase specifically in certain tissues and cells, certain tissue- and cell-specific inflammatory signals desired by a researcher can be evaluated in vitro and the progress of inflammatory changes over time without the autopsy of living animals can be identified, and thus the present invention can be used throughout the field of life science research including experimental animal science, molecular biology and the like, and can also be used as a model for efficacy evaluation of anti-inflammatory candidates in the industrial area such as new drug or health functional food development and the like.

### BRIEF DESCRIPTION OF THE DRAWINGS

FIG. 1 is a diagram showing the structure of an inflammatory signal response cassette used in the preparation of a mouse model for bio-imaging of inflammatory signals.

FIG. 1a is a schematic diagram showing the targeting vector constructed by inserting a tissue-specific NF-κB (nuclear factor kappa-light-chain-enhancer of activated B cells) reporter cassette into the mouse ROSA26 locus.

FIG. 1b is a diagram showing the NF-κB (nuclear factor kappa-light-chain-enhancer of activated B cells) reporter cassette inserted into the mouse ROSA26 locus.

FIG. 1c is a diagram showing the final gene structure prepared by inserting the NF-κ (nuclear factor kappa-light-chain-enhancer of activated B cells) reporter cassette into the mouse ROSA26 locus.

FIG. 1d is a diagram showing the results of performing Southern blot to confirm clones in which the inflammatory signal response cassette was accurately inserted into the ROSA26 locus.

FIG. 2a is a diagram showing the imaging of inflammatory signals using luciferase activity after treatment with PMA on the left ear of a mouse capable of myeloid lineage cell-specific imaging of inflammatory signals obtained by mating a mouse model for bio-imaging of inflammatory signals with a Lyz2-Cre mouse expressing Cre recombinase specifically in myeloid lineage cells.

FIG. 2b is a graph showing the measured fluorescence value of FIG. 2a.

FIG. 3a is a diagram showing the imaging of inflammatory signals using luciferase activity after colitis was induced by supplying DSS (dextran sulfate sodium salt) as drinking water to a mouse model capable of myeloid lineage cell-specific imaging of inflammatory signals.

FIG. 3b is a graph showing the measured fluorescence value of FIG. 3a.

FIG. 4 is a diagram showing the evaluation of inflammatory signals by luciferase activity test and Western blotting after macrophages were differentiated from the bone marrow of a mouse model capable of myeloid lineage cell-specific imaging of inflammatory signals and treated with LPS (lipopolysaccharide) to induce inflammation.

FIG. 4a is a graph confirming the degree of NF-κB activity in macrophages differentiated from an inflammation-induced mouse model for myeloid lineage cell-specific imaging of inflammatory signals through evaluation of luciferase activity.

FIG. 4b is a diagram confirming the degree of NF-κB activity in macrophages differentiated from an inflammation-induced mouse model for myeloid lineage cell-specific imaging of inflammatory signals through Western blotting.

FIG. 4c is a graph confirming the degree of NF-kB activity in macrophages differentiated from an inflammation-induced mouse model with LPS for myeloid lineage cell-specific imaging of inflammatory signals through evaluation of luciferase activity, and confirming that the activity was inhibited by treatment of BAY 11-7082, an NF-kB inhibitor.

FIG. 5a is a diagram showing the imaging of inflammatory signals using luciferase activity after hepatitis was induced by treating LPS (lipopolysaccharide) and D-galactosamine to a mouse model capable of hepatocyte-specific imaging of inflammatory signals obtained by mating a mouse model for bio-imaging of inflammatory signals with an Alb-Cre mouse expressing Cre recombinase specifically in hepatocytes.

FIG. 5b is a graph showing the measured fluorescence value of FIG. 5a.

### DESCRIPTION OF THE PREFERRED EMBODIMENTS

Hereinafter, the present invention is described in detail.

In an aspect of the present invention, the present invention provides a method for preparing a mouse model for bio-imaging of inflammatory signals comprising the following steps:
1) a step of preparing a targeting vector by inserting a gene cassette including a nucleotide sequence encoding NF-κB RE (nuclear factor kappa-light-chain-enhancer of activated B cells response element) and a reporter gene into a mouse ROSA26 locus;
2) a step of preparing a mouse embryonic stem cell clone by inserting the targeting vector prepared in step 1) into mouse embryonic stem cells;
3) a step of inserting the mouse embryonic stem cell clone prepared in step 2) into the blastocyst isolated from wild-type mice;
4) a step of implanting the blastocyst into which the clone of step 3) is inserted into the uterus of a surrogate mouse; and
5) a step of preparing a heterozygous mouse by mating the mouse born from the surrogate mouse of step 4) with a wild-type mouse.

Hereinafter, the method for preparing a mouse model for bio-imaging of inflammatory signals is described in detail step by step.

In the method for preparing a mouse model for bio-imaging of inflammatory signals of the present invention, step 1) is a step of preparing a targeting vector by inserting a gene cassette including a nucleotide sequence encoding NF-κB RE (nuclear factor kappa-light-chain-enhancer of activated B cells response element) and a reporter gene into a mouse ROSA26 locus

The gene cassette can include a nucleotide sequence encoding NF-κB RE and a reporter gene, preferably can sequentially include a nucleotide sequence encoding NF-κB RE, a TA promoter, a nucleotide sequence encoding luciferase, a UBC promoter, and a nucleotide sequence encoding tdTomato, but not always limited thereto.

The targeting vector can sequentially include a CAG promoter, a gene fragment in which a transcription stop codon site is located between two loxP (locus of X-over P1) sites, a nucleotide sequence encoding NF-κB RE, a TA promoter, a nucleotide sequence encoding luciferase, a UBC promoter, a nucleotide sequence encoding tdTomato, BGH poly A, and a neomycin resistance gene.

The reporter gene may be a gene encoding any one selected from the group consisting of luciferase, β-galactosidase, Green Fluorescent Protein (GFP), enhanced Green Fluorescent Protein (eGFP), mPlum, mCherry, tdTomato, mStrawberry, J-Red, DsRed, mOrange, mKO, mCitrine, Venus, YPet, enhanced yellow fluorescent protein (EYFP), Emerald, CyPet, cyan fluorescent protein (CFP), Cerulean, T-Sapphire, and alkaline phosphatase, and preferably, the reporter gene may be luciferase or tdTomato, but not always limited thereto.

In the method for preparing a mouse model for bio-imaging of inflammatory signals of the present invention, step 2) is a step of preparing a mouse embryonic stem cell clone by inserting the targeting vector prepared in step 1) into mouse embryonic stem cells.

Step 2) can further include a step of selecting a mouse embryonic stem cell clone into which the targeting vector is inserted by treating the mouse embryonic stem cell clone with neomycin and performing Southern blotting.

When neomycin, an antibiotic, is treated in the mouse embryonic stem cell clone, the clone into which the targeting vector is inserted can survive because it is resistant to neomycin, but the clone without the targeting vector cannot survive because it is not resistant to neomycin.

The Southern blot is an experimental method used in molecular biology to detect a specific DNA sequence in DNA samples. When DNA fragments are separated in order of molecular weight by electrophoresis, a DNA fragment band corresponding to 7.8 kb can be identified in clones in which the targeting vector has been correctly inserted.

In the method for preparing a mouse model for bio-imaging of inflammatory signals of the present invention, step 3) is a step of inserting the mouse embryonic stem cell clone prepared in step 2) into the blastocyst isolated from wild-type mice.

The blastocyst is a structure formed in the initial stage of development during the mammalian developmental process. The inner cell mass that forms an embryo constitutes the outer layer of the blastocyst, and it consists of the trophoblast that becomes the placenta after implantation. When the blastocyst is inserted into the uterus, it is incorporated into the inner wall of the uterus, and after the blastocyst is implanted in the uterus, late development including a gastrula stage proceeds.

In the method for preparing a mouse model for bio-imaging of inflammatory signals of the present invention, step 4) is a step of implanting the blastocyst into which the clone of step 3) is inserted into the uterus of a surrogate mouse.

The gestation period of the surrogate mouse may be 10 to 25 days after the implantation, preferably 15 to 20 days after the implantation, and more preferably 17 days after the implantation, but not always limited thereto.

In the method for preparing a mouse model for bio-imaging of inflammatory signals of the present invention, step 5) is a step of preparing a heterozygous mouse by mating the mouse born from the surrogate mouse of step 4) with a wild-type mouse.

The wild-type mouse mated with the mouse born from the surrogate mother may be a C57BL/6 mouse, but not always limited thereto.

In another aspect of the present invention, the present invention provides a mouse model for bio-imaging of inflammatory signals prepared by the method for preparing a mouse model for bio-imaging of inflammatory signals.

The mouse model for bio-imaging of inflammatory signals can be mated with a genetically engineered mouse expressing Cre recombinase specifically in certain tissues and cells to prepare a mouse in which cell or tissue specific inflammatory signals are expressed.

The genetically engineered mouse expressing Cre recombinase specifically in certain tissues and cells may be any one selected from the group consisting of MMTV(mouse mammary tumor virus promoter)-Cre mouse, Pdx1(pancreatic and duodenal homeobox 1)-Cre mouse, Foxp3(forkhead box P3)-Cre mouse, CD4(cluster of differentiation 4)-Cre mouse, CD8(cluster of differentiation 8)-Cre mouse, CD11c(cluster of differentiation 11c)-Cre mouse, Vil(villin 1)-Cre mouse, Alb(albumin)-Cre mouse, AQ(adipoq)-Cre mouse, AP2(adipocyte protein 2)-CreERT2(Cre recombinase fused to a mutant estrogen ligand-binding domain(ERT2)) mouse, Lyz2(lysozyme 2, LysM)-Cre mouse, Ins2(insulin 2)-Cre mouse and DAT(dopamine transporter)-Cre mouse. In a specific embodiment of the present invention, the mouse model for bio-imaging of inflammatory signals was mated with the Lyz2-Cre mouse and the Alb-Cre mouse to construct a myeloid lineage cell-specific and hepatocyte-specific mouse model, but not always limited thereto.

In another aspect of the present invention, the present invention provides a method for screening an anti-inflammatory substance comprising the following steps:
1) a step of treating a test substance to a mouse prepared by mating the mouse model of claim 7 with a genetically engineered mouse expressing Cre recombinase specifically in certain cells and tissues;
2) a step of measuring the activity level of NF-κ (nuclear factor kappa-light-chain-enhancer of activated B cells) in the mouse treated with the test substance; and
3) a step of selecting a test substance that reduced the activity level of NF-κ compared to the control group.

Hereinafter, the method of screening an anti-inflammatory substance is described in detail step by step.

In the method for screening an anti-inflammatory substance of the present invention, step 1) is a step of treating a test substance to a mouse prepared by mating the mouse model for bio-imaging of inflammatory signals with a genetically engineered mouse expressing Cre recombinase specifically in certain cells and tissues.

The mouse model for bio-imaging of inflammatory signals can be mated with a genetically engineered mouse expressing Cre recombinase specifically in certain cells and tissues to prepare a mouse in which cell or tissue specific inflammatory signals are expressed.

The genetically engineered mouse expressing Cre recombinase specifically in certain cells and tissues may be any one selected from the group consisting of MMTV(mouse mammary tumor virus promoter)-Cre mouse, Pdx1(pancreatic and duodenal homeobox 1)-Cre mouse, Foxp3(forkhead box P3)-Cre mouse, CD4(cluster of differentiation 4)-Cre mouse, CD8(cluster of differentiation 8)-Cre mouse, CD11c(cluster of differentiation 11c)-Cre mouse, Vil(villin 1)-Cre mouse, Alb(albumin)-Cre mouse, AQ(adipoq)-Cre mouse, AP2(adipocyte protein 2)-CreERT2(Cre recombinase fused to a mutant estrogen ligand-binding domain(ERT2)) mouse, Lyz2(lysozyme 2, LysM)-Cre mouse, Ins2(insulin 2)-Cre mouse and DAT(dopamine transporter)-Cre mouse. In a specific embodiment of the present invention, the mouse model for bio-imaging of inflammatory signals was mated with the Lyz2-Cre mouse and the Alb-Cre mouse to construct a myeloid lineage cell-specific and hepatocyte-specific mouse model, but not always limited thereto.

In the method for screening an anti-inflammatory substance of the present invention, step 2) is a step of measuring the activity level of NF-κB (nuclear factor kappa-light-chain-enhancer of activated B cells) in the mouse treated with the test substance.

The activity level of NF-κB can be measured by the expression level of any one reporter gene selected from the group consisting of luciferase, β-galactosidase, Green Fluorescent Protein (GFP), enhanced Green Fluorescent Protein (eGFP), mPlum, mCherry, tdTomato, mStrawberry, J-Red, DsRed, mOrange, mKO, mCitrine, Venus, YPet, enhanced yellow fluorescent protein (EYFP), Emerald, CyPet, cyan fluorescent protein (CFP), Cerulean, T-Sapphire, and alkaline phosphatase, and preferably by the expression level of luciferase or tdTomato, but not always limited thereto.

Hereinafter, the present invention will be described in detail by the following examples and experimental examples.

However, the following examples and experimental examples are only for illustrating the present invention, and the contents of the present invention are not limited thereto.

### <Example 1> Preparation of Mouse for Bio-Imaging of Inflammatory Signals and Confirmation Thereof

<1-1> Preparation of Mouse for Bio-Imaging of Inflammatory Signals

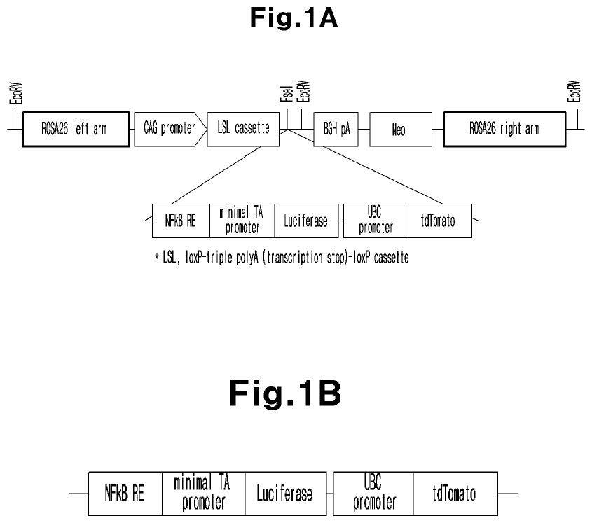

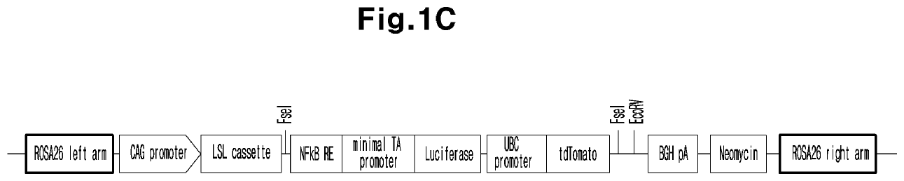

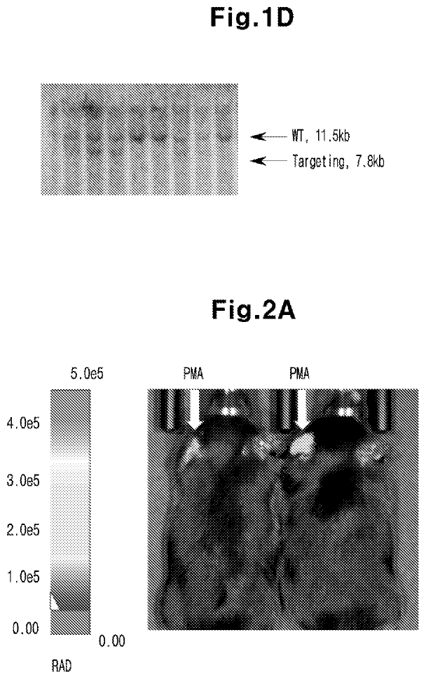

To construct a mouse model for bio-imaging of inflammatory signals, a cassette responding to inflammatory signals sequentially containing a nucleotide sequence encoding NF-κB RE (nuclear factor kappa-light-chain-enhancer of activated B cells response element), a minimal TA promoter, a nucleotide sequence encoding luciferase, a UBC promoter, and a nucleotide sequence encoding tdTomato was prepared (FIG. 1b). A targeting vector that can be inserted into a mouse ROSA26 locus was prepared using the cassette (FIG. 1c). Finally, the targeting vector consisted of a left nucleotide sequence of the ROSA26 locus, a tissue-specific inflammatory signal response cassette, and a right nucleotide sequence of the ROSA26 locus.

After inserting the targeting vector prepared above into mouse embryonic stem cells and selecting with neomycin, the clones in which the inflammatory signal response cassette was accurately inserted into the ROSA26 locus were confirmed by Southern blotting of Example <1-2> (FIG. 1d). The clone into which the targeting vector was correctly inserted showed a DNA fragment band corresponding to 7.8 kb in Southern blotting, and a DNA fragment band corresponding to 11.5 kb appeared in the wild-type vector.

The mouse embryonic stem cell clone obtained by Southern blotting was inserted into a blastocyst isolated from a wild-type C57BL/6 female, and transplanted into the uterus of a surrogate mother. About 17 days after the transplantation, the offsprings were born, and the offsprings born from the inserted mouse embryonic stem cells had chimeric fur color. The obtained chimera mouse was crossed with a wild-type C57BL/6 mouse to secure a heterozygous/hetero mouse (heterozygote).

<1-2> Confirmation of Mouse Gene by Southern Blotting

Southern blotting was performed to confirm the gene of the heterozygous mouse obtained in Example <1-1>.

Particularly, genomic DNA was extracted from mouse embryonic stem cells, and the DNA was digested with EcoRV. After electrophoresis of 10 to 20 μg of DNA on a 0.8% agarose gel, the agarose gel was transferred to a transfer vessel for Southern blotting. A nylon membrane was placed on the agarose gel, Whatman 3MM paper was placed thereon, a sufficient amount of towel paper was placed thereon again, and a solution of 0.5 M NaOH was poured on the bottom. After 12 hours of transfer, the nylon membrane was separated, and DNA was crosslinked on the surface of the nylon membrane using a UV transilluminator (wavelength: 254 nm). Then, a DNA fragment of 300 to 400 bp that can attach to the mouse ROSA26 locus was prepared, and a probe for Southern blotting was prepared using an isotope and a random priming kit. A nylon membrane, a DNA probe, and a hybridization solution were put in a hybridization bottle, followed by incubated in a 65° C. hybridization oven for 12 hours. After attaching the DNA probe to the nylon membrane for a sufficient amount of time, the probe not attached to the nylon membrane was wiped off using a washing solution. After the washing was completed, it was exposed to a phosphoimager (GE FLA7000) plate for 24 to 48 hours. After the exposure was completed, the plate was scanned using a phosphoimager (GE FLA7000).

As a result, only 11.5 kb-sized DNA fragments were confirmed in wild-type mice, and both 11.5 kb and 7.8 kb-sized DNA fragments were confirmed in heterozygous mice (FIG. 1d).

### Example 2> Establishment of Mouse for Myeloid Lineage Cell-Specific Bio-Imaging of Inflammatory Signals and Confirmation Thereof

<2-1> Establishment of Mouse for Myeloid Lineage Cell-Specific Bio-Imaging of Inflammatory Signals

Since the mouse for bio-imaging of inflammatory signals prepared in Example <1-1> contains a stop codon marked by a loxP site on the gene cassette structure, the mouse can express luciferase according to the activity of NF-κB, an inflammation-related factor, only when the stop codon is removed by mating with a mouse expressing Cre recombinase. Accordingly, a mouse model capable of myeloid lineage cell-specific bio-imaging of inflammatory signals was established by mating the mouse above with a Lyz2 (lysozyme 2)-Cre mouse expressing Cre recombinase specifically in myeloid-lineage cells.

<2-2> PMA-Induced Ear Edema Model

To confirm imaging of inflammatory signals in the mouse model established in Example <2-1>, ear edema was induced by treatment with PMA (phorbol 12-myristate 13-acetate), an inflammatory factor.

Particularly, the left ear of the mouse was treated with 2 μg/40 [symbol] of PMA diluted in ethanol using a micropipette, and the right ear was treated with the same amount of ethanol (40 [symbol] ). After 24 hours, the left ear was treated with 2 μg of PMA, and the right ear was treated with the same amount of ethanol (40 [symbol] ). After another 24 hours, 100 [symbol] of D-luciferin (15 mg/ml in PBS) was intraperitoneally administered using a 1 ml syringe, and luciferase signals were measured in the ears of the mouse under respiratory anesthesia with isoflurane.

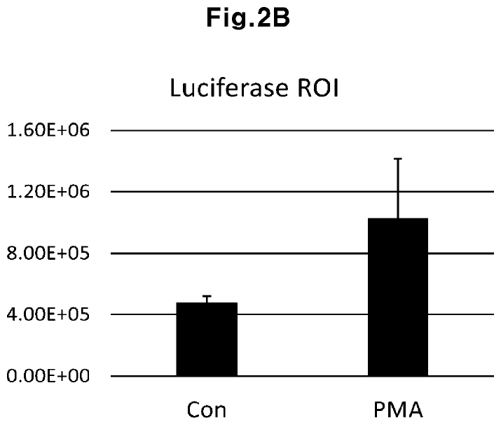

As a result, it was confirmed that the luciferase signal was specifically strong in the left ear induced inflammation by PMA compared to the right ear treated with only ethanol (FIGS. 2a and 2b).

<2-3> DSS-Induced Colitis Model

Colitis was induced by DSS (dextran sulfate sodium salt) in the mouse model established in Example <2-1>, and it was confirmed whether bio-imaging of inflammatory signals over time was possible.

Particularly, 6 g of DSS was added to sterilized tertiary distilled water and stirred for 60 minutes to prepare a 3% DSS aqueous solution. Before supplying 3% DSS, 100 [symbol] of D-luciferin (15 mg/ [symbol] in PBS) was intraperitoneally administered to each mouse using a 1 [symbol] syringe, and luciferase signals were measured in the abdomen under respiratory anesthesia with isoflurane. After the measurement, the 3% DSS aqueous solution was supplied as drinking water to the mice. While maintaining DSS supply, 100 [symbol] of D-luciferin (15 mg/ [symbol] in PBS) was intraperitoneally administered using a 1 [symbol] syringe on the days after 3 and 5 days after the start of DSS supply, and luciferase signals were measured in the abdomen.

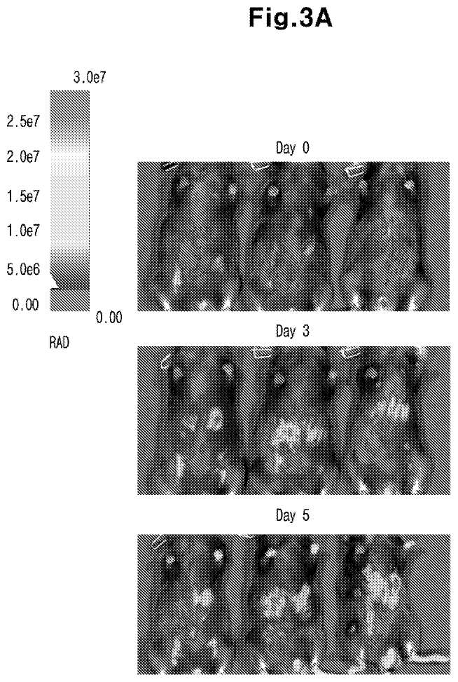

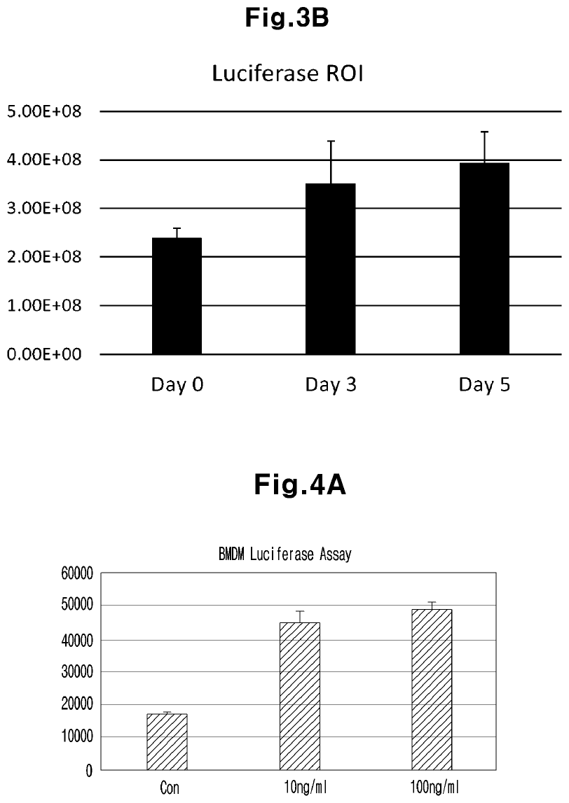

As a result, it was confirmed that the luciferase signal was strongly expressed in the large intestine region of the abdomen over time after the DSS supply was started (FIGS. 3a and 3b).

<2-4> Bone Marrow-Derived Macrophage Differentiation and LPS Treatment

Macrophages, mononuclear cells, derived from the bone marrow of the mouse model established in Example <2-1> were differentiated, luciferase activity was confirmed, and NF-κ activity and luciferase protein expression were confirmed by Western blotting. In addition, 20 uM BAY 11-7082 (NF-kB inhibitor) was treated simultaneously with LPS in the same manner as above to evaluate the luciferase activity.

Particularly, the mouse was euthanized with CO₂, the femur and tibia were obtained, and the bone marrow cavity was exposed. Then, RPMI 1640 culture medium (10% FBS, 1% penicillin, stretptomycin) containing M-CSF was passed through using a 1 ml syringe, and the contents were collected to make 6 ml of the culture medium. Thereafter, the contents were transferred to a Petri dish and cultured for 3 days at 37° C. under 5% CO₂ conditions, and then the medium was replaced with fresh RPMI 1640 culture medium. After 3 days, the culture medium was removed from the Petri dish and washed with PBS (phosphate buffered saline). Then, the cells were detached using a cell dissociation kit, diluted in RPMI 1640 culture medium, and then distributed in a white 96 well plate at the density of 60,000 cells/well, followed by culture. After 24 hours of culture, the experimental group was treated with LPS (lipopolysaccharide) at the concentrations of 10 ng/ml and 100 ng/ml. After 6 hours, the culture medium was removed, and 100 μl of HBSS (Hanks' Balanced Salt solution) containing D-luciferin was added to each well of the plate, and then the luciferase activity was evaluated.

For Western blotting, the detached cells were diluted in RPMI 1640 culture medium, and then distributed in a 6 well plate at the density of 70,000 cells/well, followed by culture. After 24 hours of culture, the experimental group was treated with LPS at the concentrations of 10 ng/ml and 100 ng/ml for 6 hours. The culture medium was removed and homogenized with a buffer containing 20 mM Tris-HCl (pH 7.4), 1% Triton X-100, 15 mM NaCl and protease inhibition cocktail, and centrifuged at 4° C., 13,000 g for 20 minutes. Then, SDS-PAGE (sodium dodecyl sulfate-polyacrylamide gel electrophoresis) was performed using the supernatant to separate proteins. The separated proteins were transferred to a PVDF membrane and blocked with TBST buffer (Tris-Buffered Saline, 0.1% TWEEN 20) containing 5% skim milk powder. The membrane was reacted with a primary antibody for 12 hours at 4° C., then reacted with a secondary antibody containing horseradish peroxidase for 1 hour, and treated with ECL to detect signals.

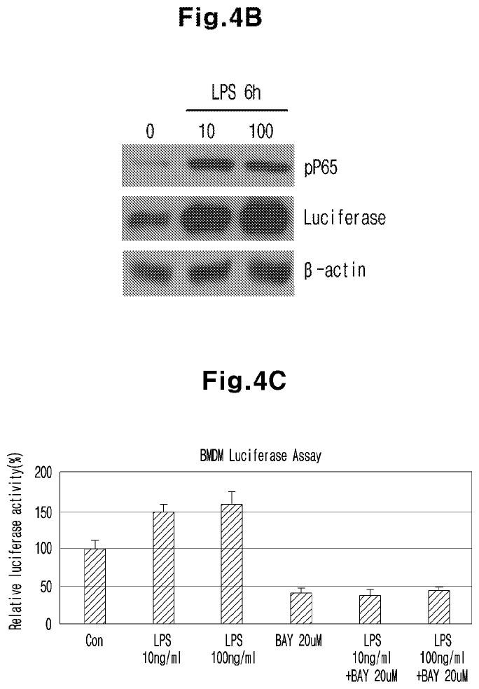

As a result, it was confirmed that the luciferase signal activity was increased by LPS dose-dependently in macrophages of the euthanized mouse (FIG. 4a), and that the luciferase expression was increased in the same way as the in vitro inflammatory signals measurement result by Western blotting (FIG. 4b). In addition, it was confirmed that the increased luciferase signal activity by LPS dose-dependently was suppressed by the treatment of BAY 11-7082, a NF-kB inhibitor (FIG. 4c).

### <Example 3> Establishment of Mouse for Hepatocyte-Specific Bio-Imaging of Inflammatory Signals and Confirmation Thereof

<3-1> Establishment of Mouse for Hepatocyte-Specific Bio-Imaging of Inflammatory Signals

Similar to the mouse model established in Example <2-1>, a mouse model capable of hepatocyte-specific bio-imaging was established by mating the mouse prepared in Example <1-1> with a mouse expressing Cre recombinase specifically in hepatocytes.

<3-2> LPS/D-Galactosamine-Induced Hepatitis Model

In order to confirm whether bio-imaging of inflammatory signals was possible in the mouse model established in Example <3-1>, hepatitis was induced by using LPS (lipopolysaccharide) and D-galactosamine in combination, and luciferase signals were evaluated.

Particularly, 100 [symbol] of D-luciferin (15 mg/ [symbol] in PBS) was intraperitoneally administered to each mouse using a 1 [symbol] syringe, and luciferase signals were measured in the abdomen under respiratory anesthesia with isoflurane. Then, LPS and D-galactosamine diluted in PBS (phosphate buffered saline) were intraperitoneally administered to each mouse at doses of 15 μg/kg and 350 mg/kg, respectively. After 4 hours, 100 [symbol] of D-luciferin (15 mg/ [symbol] in PBS) was intraperitoneally administered to each mouse in the same manner as above, and luciferase signals were measured.

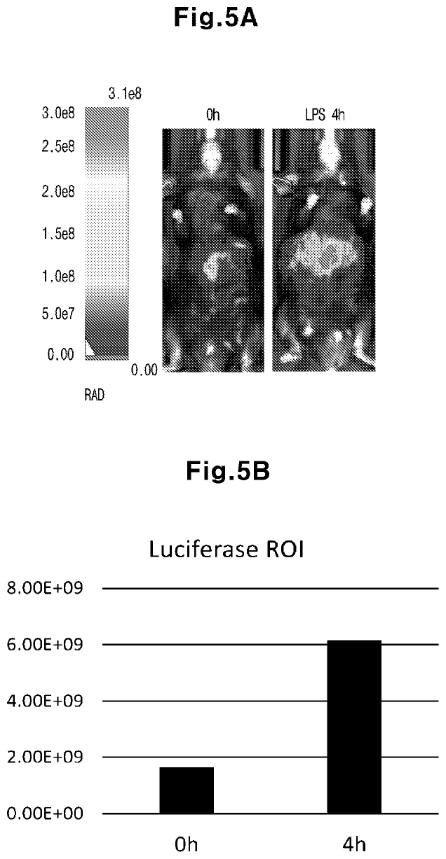

As a result, it was confirmed that inflammatory signals were increased in the liver after the administration of LPS/D-galactosamine (FIGS. 5a and 5b).

1. A method for preparing a mouse model for bio-imaging of inflammatory signals comprising the following steps:
1) a step of preparing a targeting vector by inserting a gene cassette including a nucleotide sequence encoding NF-κB RE (nuclear factor kappa-light-chain-enhancer of activated B cells response element) and a reporter gene into a mouse ROSA26 locus;
2) a step of preparing a mouse embryonic stem cell clone by inserting the targeting vector prepared in step 1) into mouse embryonic stem cells;
3) a step of inserting the mouse embryonic stem cell clone prepared in step 2) into the blastocyst isolated from a wild-type mouse;
4) a step of implanting the blastocyst into which the clone of step 3) is inserted into the uterus of a surrogate mouse; and
5) a step of preparing a heterozygous mouse by mating the mouse born from the surrogate mouse of step 4) with a wild-type mouse.

2. The method for preparing a mouse model for bio-imaging of inflammatory signals according to claim 1, wherein the targeting vector of step 1) sequentially includes the following nucleotide sequences:
(a) a CAG promoter;
(b) a gene fragment in which a transcription stop codon site is located between two loxP (locus of X-over P1) sites;
(c) a nucleotide sequence encoding NF-κB RE;
(d) a TA promoter;
(e) a nucleotide sequence encoding luciferase;
(f) a UBC promoter;
(g) a nucleotide sequence encoding tdTomato;
(h) BGH poly A; and
(i) a neomycin resistance gene.

3. The method for preparing a mouse model for bio-imaging of inflammatory signals according to claim 1, wherein the reporter gene of step 1) is a gene encoding any one selected from the group consisting of luciferase, β-galactosidase, Green Fluorescent Protein (GFP), enhanced Green Fluorescent Protein (eGFP), mPlum, mCherry, tdTomato, mStrawberry, J-Red, DsRed, mOrange, mKO, mCitrine, Venus, YPet, enhanced yellow fluorescent protein (EYFP), Emerald, CyPet, cyan fluorescent protein (CFP), Cerulean, T-Sapphire, and alkaline phosphatase.

4. The method for preparing a mouse model for bio-imaging of inflammatory signals according to claim 1, wherein the step 2) further includes a step of selecting a mouse embryonic stem cell clone into which the targeting vector is inserted by treating the mouse embryonic stem cell clone with neomycin and performing Southern blotting.

5. The method for preparing a mouse model for bio-imaging of inflammatory signals according to claim 1, wherein the gestation period of the surrogate mouse of step 4) is 15 to 20 days after the implantation.

6. The method for preparing a mouse model for bio-imaging of inflammatory signals according to claim 1, wherein the wild-type mouse of step 5) is a C57BL/6 mouse.

7. A mouse model for bio-imaging of inflammatory signals prepared by the method for preparing a mouse model for bio-imaging of inflammatory signals of claim 1.

8. The mouse model for bio-imaging of inflammatory signals according to claim 7, wherein the mouse model is mated with a genetically engineered mouse expressing Cre recombinase specifically in certain cells or tissues to prepare a mouse in which cell or tissue specific inflammatory signals are expressed.

9. The mouse model for bio-imaging of inflammatory signals according to claim 8, wherein the genetically engineered mouse expressing Cre recombinase specifically in certain cells or tissues is any one selected from the group consisting of MMTV(mouse mammary tumor virus promoter)-Cre mouse, Pdx1(pancreatic and duodenal homeobox 1)-Cre mouse, Foxp3(forkhead box P3)-Cre mouse, CD4(cluster of differentiation 4)-Cre mouse, CD8(cluster of differentiation 8)-Cre mouse, CD11c(cluster of differentiation 11c)-Cre mouse, Vil(villin 1)-Cre mouse, Alb(albumin)-Cre mouse, AQ(adipoq)-Cre mouse, AP2(adipocyte protein 2)-CreERT2(Cre recombinase fused to a mutant estrogen ligand-binding domain (ERT2)) mouse, Lyz2(lysozyme 2, LysM)-Cre mouse, Ins2(insulin 2)-Cre mouse and DAT(dopamine transporter)-Cre mouse.

10. A method for screening of an anti-inflammatory substance comprising the following steps:
1) a step of treating a test substance to a mouse prepared by mating the mouse model of claim 7 with a genetically engineered mouse expressing Cre recombinase specifically in certain cells or tissues;
2) a step of measuring the activity level of NF-κB (nuclear factor kappa-light-chain-enhancer of activated B cells) in the mouse treated with the test substance; and
3) a step of selecting a test substance that reduced the activity level of NF-κB compared to the control group.

11. The method for screening of an anti-inflammatory substance according to claim 10, wherein the genetically engineered mouse expressing Cre recombinase specifically in certain cells or tissues of step 1) is any one selected from the group consisting of MMTV(mouse mammary tumor virus promoter)-Cre mouse, Pdx1(pancreatic and duodenal homeobox 1)-Cre mouse, Foxp3(forkhead box P3)-Cre mouse, CD4(cluster of differentiation 4)-Cre mouse, CD8(cluster of differentiation 8)-Cre mouse, CD11c(cluster of differentiation 11c)-Cre mouse, Vil(villin 1)-Cre mouse, Alb(albumin)-Cre mouse, AQ(adipoq)-Cre mouse, AP2(adipocyte protein 2)-CreERT2(Cre recombinase fused to a mutant estrogen ligand-binding domain(ERT2)) mouse, Lyz2(lysozyme 2, LysM)-Cre mouse, Ins2(insulin 2)-Cre mouse and DAT(dopamine transporter)-Cre mouse.

12. The method for screening an anti-inflammatory substance according to claim 10, wherein the activity level of NF-κB of step 2) is measured by the expression level of any one reporter gene selected from the group consisting of luciferase, β-galactosidase, Green Fluorescent Protein (GFP), enhanced Green Fluorescent Protein (eGFP), mPlum, mCherry, tdTomato, mStrawberry, J-Red, DsRed, mOrange, mKO, mCitrine, Venus, YPet, enhanced yellow fluorescent protein (EYFP), Emerald, CyPet, cyan fluorescent protein (CFP), Cerulean, T-Sapphire, and alkaline phosphatase
